(19) United States
(12) Patent Application Publication
(10) Pub. No.: US 2019/0159828 A1
(43) Pub. Date: May 30, 2019

(54) ACTIVE ELECTRODE ASSEMBLY FOR AN ELECTROSURGICAL DEVICE

(71) Applicant: MESHIL A.M.O.H. AL-JARBA, SAFAT (KW)

(72) Inventor: MESHIL A.M.O.H. AL-JARBA, SAFAT (KW)

(21) Appl. No.: 16/160,967

(22) Filed: Oct. 15, 2018

Related U.S. Application Data
(60) Provisional application No. 62/592,141, filed on Nov. 29, 2017.

(51) Int. Cl. A61B 18/14

(52) U.S. Cl. CPC A61B 18/14; A61B 2018/00083

(57) ABSTRACT

The active electrode assembly for an electrosurgical device includes an electrically conductive shaft having opposed proximal and distal ends, the proximal end being adapted for connection to a conventional electrosurgical device. An electrically insulating sheath partially covers the distal end of the electrically conductive shaft. The electrically insulating sheath has a cut-out portion or recess formed therein, such that a portion of the distal end of the electrically conductive shaft is exposed therethrough. The exposed portion of the distal end defines an application surface for applying electrical current to the patient's tissue. The area surrounding the application surface remains covered by the electrically insulating sheath, thus providing electrically insulating protection for both the patient and the surgeon.

### CROSS-REFERENCE TO RELATED APPLICATION

This application claims the benefit of U.S. Provisional Patent Application Ser. No. 62/592,141, filed on Nov. 29, 2017.

### BACKGROUND

### 1. Field

The disclosure of the present patent application relates to electrosurgery, and particularly to an active electrode assembly for an electrosurgical device which provides electrically insulating protection for surrounding tissues.

### 2. Description of the Related Art

Electrosurgical devices have become increasingly common, particularly for the modification, sculpting, resection, removal or vaporization of tissue. Such devices are typically configured for coagulation, cauterization or hemostasis purposes, or are utilized for thermal treatment of normal and tumorous tissues. FIGS. 2 and 3 illustrate a typical electrosurgical system 100, including an electrosurgical power supply 102, an electrosurgical ablator 104 (with electrical cord 106), and a dispersive (return) electrode 108 (with electrical cord 110). The conventional electrosurgical ablator 104 has a proximal portion 112, which forms a handle, and a proximal end 114, from which extends the electrical cord 106, along with a distal end 118 which attaches to a proximal end 120 of elongated distal portion 122. The distal portion 122 includes a distal end element 124 (i.e., the active electrode) and a tubular portion 126. The tubular portion 126 has a proximal end 128 and a distal end 130. Buttons 132 and 134 control the power (typically RF power) applied to the device. The active electrode 124 is typically in the form of a very thin strip of metal.

In use, the electrosurgical device 104 applies a high-frequency (typically radio frequency) alternating polarity electrical current to biological tissue in order to cut, coagulate, desiccate, or fulgurate the tissue. This electrical current is applied to the tissue through distal end element 124. Such electrosurgical devices are frequently used during surgical operations to help in preventing blood loss. By using radio frequency (RF) alternating current to heat the tissue by RF-induced intracellular oscillation of ionized molecules, an elevation of intracellular temperature results. When the intracellular temperature reaches 60° C., instantaneous cell death occurs. If tissue is heated to 60-99° C., the simultaneous processes of tissue desiccation (dehydration) and protein coagulation occur. Appropriately applied with electrosurgical forceps, desiccation and coagulation result in the occlusion of blood vessels and halting of bleeding. The process of vaporization can be used to ablate tissue targets, or, by linear extension, used to transect or cut tissue. While the processes of vaporization/cutting and desiccation/coagulation are best accomplished with relatively low voltage, continuous or near continuous waveforms, the process of fulguration is performed with relatively high voltage modulated waveforms. Fulguration is a superficial type of coagulation, typically created by arcing modulated high voltage current to tissue that is rapidly desiccated and coagulated. The continued application of current to this highly impedant tissue results in resistive heating and the achievement of very high temperatures, specifically enough to cause breakdown of the organic molecules to sugars and even carbon.

Radio frequency (RF) electrosurgery is performed using a RF electrosurgical generator, such as electrosurgical power supply 102, and a handpiece that includes one or two electrodes. FIG. 2 illustrates a bipolar electrosurgical system, including both an active electrode and also a dispersive (return) electrode 108. The monopolar portion of the instrument (i.e., the active electrode 124), when energized, requires the application of dispersive electrode 108 elsewhere on the patient's body to disperse the RF current, thereby preventing thermal injury to the underlying tissue.

As noted above, a typical active electrode may be in the form of a simple, thin strip of metal. Application of the active electrode to only the desired area relies solely on the precision of the surgeon using the electrosurgical device. Unfortunately, due to the precise nature of many electrosurgical procedures, adjacent areas of tissue may be inadvertently burned or vaporized. Additionally, it is possible that the surgeon may accidentally bring one or more of his or her fingers too close to the active electrode, resulting in accidental injury to the surgeon during the procedure. Thus, an active electrode assembly for an electrosurgical device solving the aforementioned problems is desired.

### SUMMARY

The active electrode assembly for an electrosurgical device includes an electrically conductive shaft having opposed proximal and distal ends, the proximal end being adapted for connection to a conventional electrosurgical device. An electrically insulating sheath partially covers the distal end of the electrically conductive shaft. The electrically insulating sheath has a cut-out portion or recess formed therein, such that a portion of the distal end of the electrically conductive shaft is exposed therethrough. The exposed portion of the distal end defines an application surface for applying electrical current to the patient's tissue. The area surrounding the application surface remains covered by the electrically insulating sheath, thus providing electrically insulating protection for both the patient and the surgeon.

In an alternative embodiment, the active electrode assembly for an electrosurgical device includes an electrically conductive shaft having opposed proximal and distal ends, the proximal end being adapted for connection to an electrosurgical device. The electrically insulating sheath in this embodiment at least partially covers the distal end of the electrically conductive shaft. A curved portion of the distal end extends from an exterior face of the electrically insulating sheath and is in electrical communication with the electrically conductive shaft. Only the curved portion of the distal end is exposed and adapted for applying electrical current to a patient's tissue for electrocoagulation procedures and the like. It should be understood that the overall configuration, shape and relative dimensions of the active electrode assembly for an electrosurgical device may be varied dependent upon the particular electrosurgical procedure. For example, some electrosurgical procedures may require the distal end of the electrically conductive shaft to be substantially straight, while others may require the distal end to be curved.

These and other features of the present disclosure will become readily apparent upon further review of the following specification and drawings.

### BRIEF DESCRIPTION OF THE DRAWINGS

FIG. 1 is a perspective view of an active electrode assembly for an electrosurgical device.

FIG. 2 illustrates a conventional prior art electrosurgical system.

FIG. 3 is a perspective view of a conventional prior art electrosurgical ablator used with the electrosurgical system of FIG. 2.

FIG. 4 is a perspective view of an alternative embodiment of the active electrode assembly for an electrosurgical device.

FIG. 5 is a perspective view of another alternative embodiment of the active electrode assembly for an electrosurgical device.

FIG. 6 is a perspective view of still another alternative embodiment of the active electrode assembly for an electrosurgical device.

Similar reference characters denote corresponding features consistently throughout the attached drawings.

### DETAILED DESCRIPTION OF THE PREFERRED EMBODIMENTS

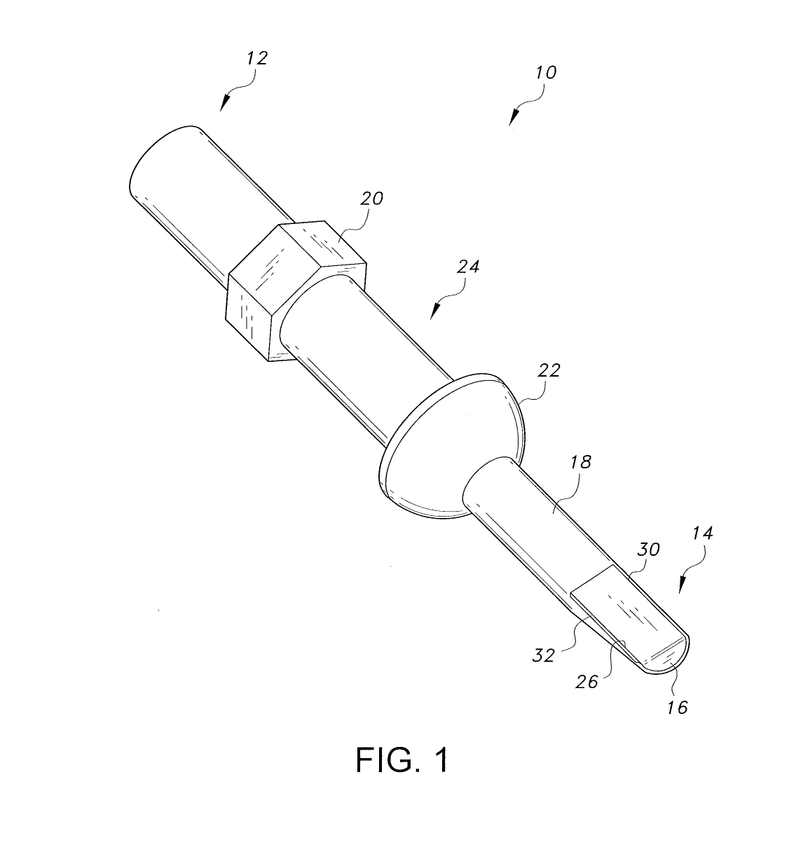

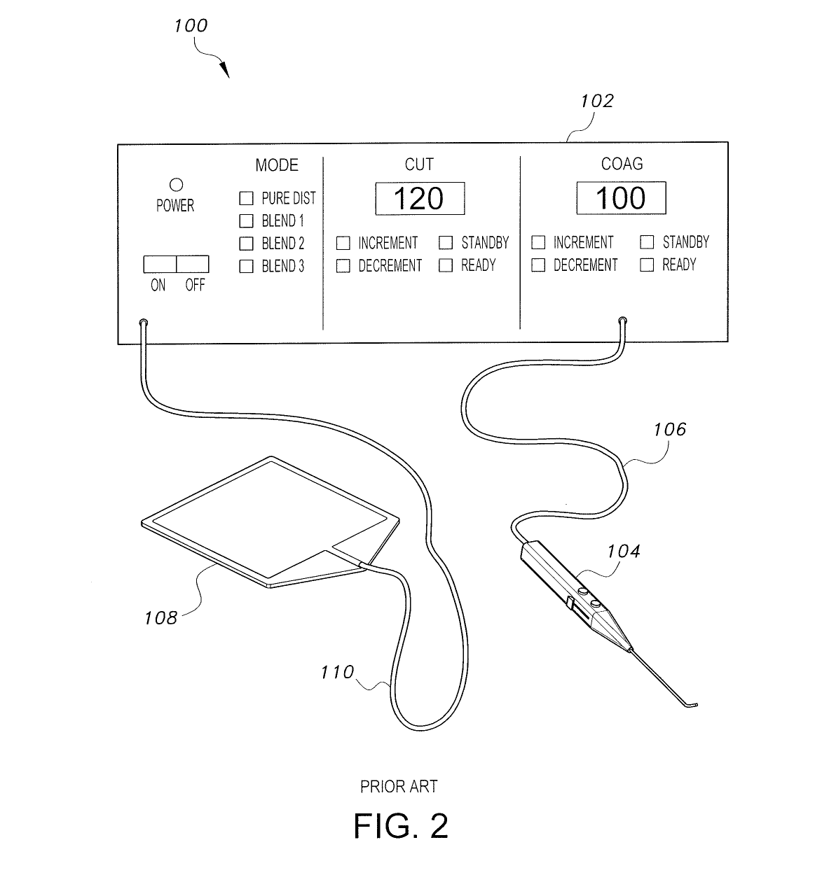

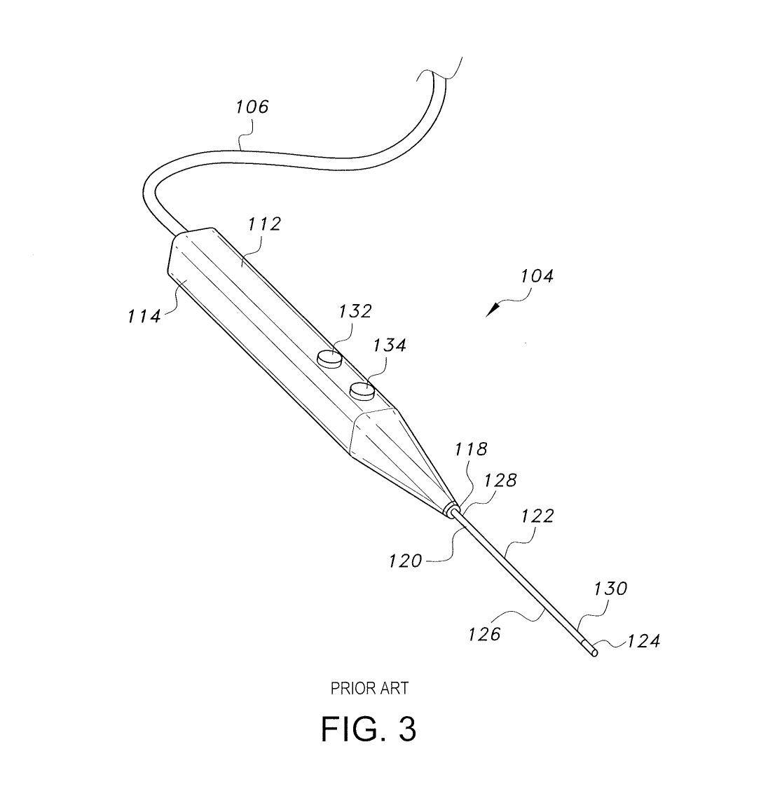

FIG. 1 shows an active electrode assembly for an electrosurgical device 10. The active electrode assembly 10 includes a proximal end 12 configured for mounting to the distal end of an electrosurgical ablator, e.g., the distal end 118 of the conventional electrosurgical ablator 104 of FIGS. 2 and 3. When mounted to the ablator 104, the active electrode assembly for an electrosurgical device 10 can be used for tissue removal in electrosurgical procedures as described above with respect to electrosurgical ablator 100 of FIGS. 2 and 3. The active electrode assembly for an electrosurgical device 10 includes a distal end 14, which acts as the electrically active surface of the active electrode 10. Although shown as having a pair of flanges 20, 22 mounted on a central portion 24, it should be understood that the overall configuration of the active electrode 10 is shown for exemplary purposes only, and that the general shape, contouring and relative dimensions may be varied.

As in a conventional active electrode, the proximal end 12, the distal end 14, and the central portion 24 of the active electrode 10 may be formed from metal or any other suitable electrically conductive material. However, as shown in FIG. 1, the active electrode assembly 10 includes an insulating sheath 18 formed from an electrically insulating material. As shown, the insulating sheath 18 partially covers the distal end 14 and has a cut-out portion 26 or recess exposing an application surface 16 of the electrode 10. Thus, in use, only the application surface 16 (i.e., the portion of the distal end 14 exposed through the cut-out portion 26 of the insulating sheath 18) is exposed for applying the RF alternating polarity electrical current to the patient's tissue. As shown, the area surrounding the application surface 16 remains covered by the electrically insulating sheath 18, thus providing protection for the patient's tissue directly adjacent the intended application site, as well as providing protection for the surgeon's fingers.

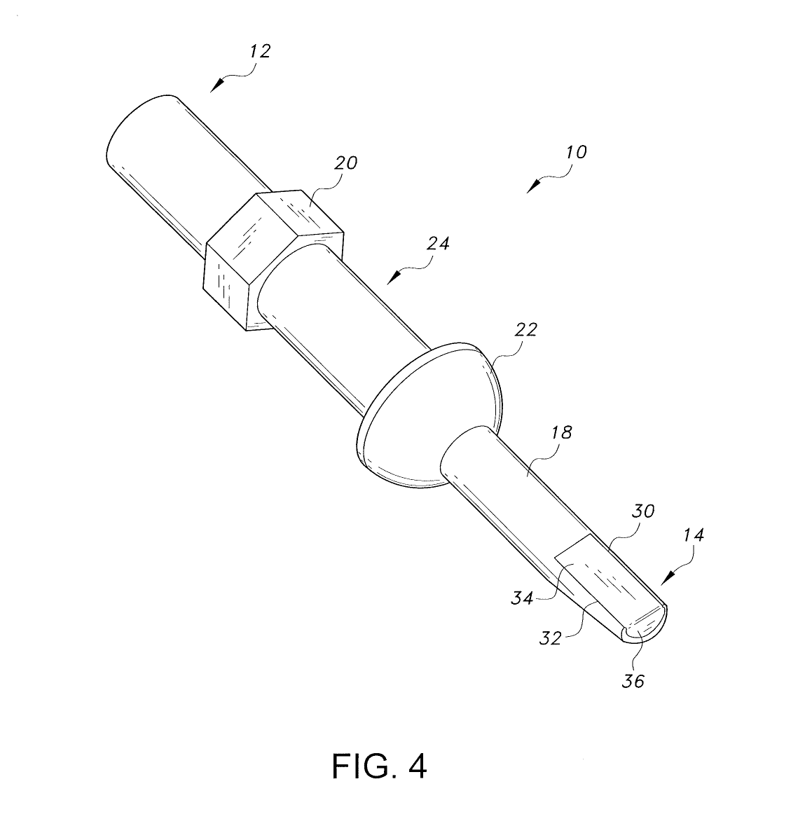

It should be understood that the contouring and relative dimensions of the cut-out portion 26 may be varied. For example, in FIG. 1, the cut-out portion 26 is sized and shaped such that one lateral side 30 of the application surface 16 is covered by insulating sheath 18, but the opposed lateral side 32 is free and exposed. It should be understood that the opposite configuration may also be used and that the configuration of FIG. 1 is shown for exemplary purposes only. As another example, in FIG. 4, both of the opposed lateral sides 30, 32 are covered by insulating sheath 18, leaving only the upper and forward surfaces 34, 36, respectively, free and exposed.

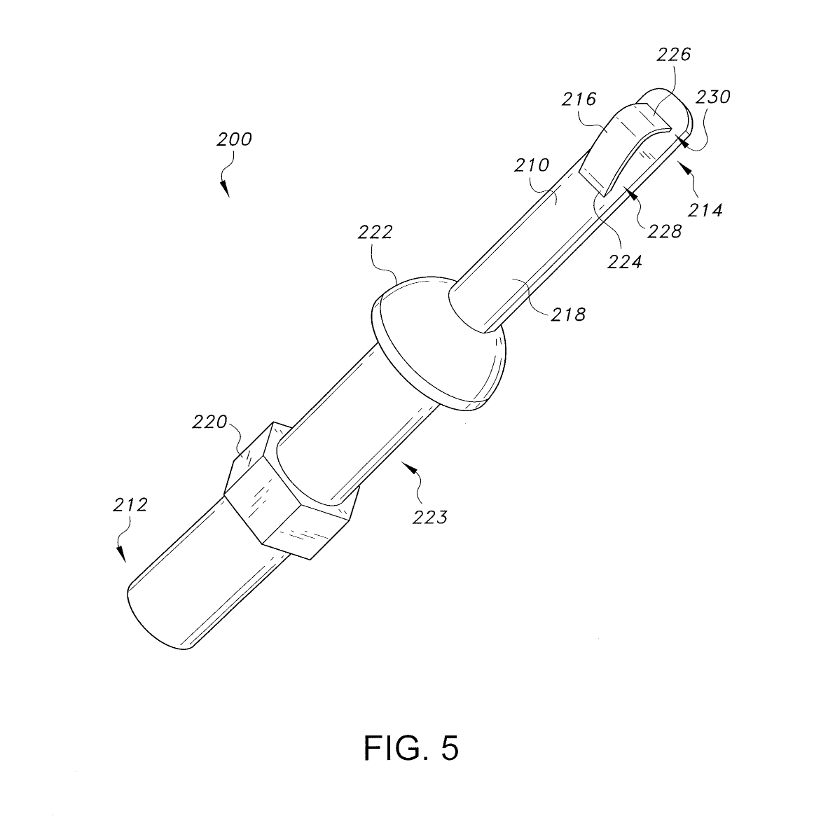

FIG. 5 shows an alternative active electrode assembly for an electrosurgical device 200, which may be used for electrocoagulation or the like. Similar to the previous embodiment, the active electrode assembly for an electrosurgical device 200 is provided in the form of an electrically conductive shaft having opposed proximal and distal ends 212, 214, respectively, and central portion 223 extending between the proximal and distal ends 212, 214. The proximal end 212 is adapted for connection to an electrosurgical device. For example, the proximal end 212 may be mounted to the electrosurgical ablator 104 of FIGS. 2 and 3.

Like the active electrode assembly 10, the proximal end 212, the distal end 214 and the central portion 223 are formed from metal or any other suitable electrically conductive material. An electrically insulating sheath 218 at least partially covers the distal end 214, as shown. Unlike the distal end 14 of the active electrode assembly 10, however, the distal end 214 of the active electrode assembly 200 includes an electrically conductive arcuate or curved portion 216 extending from an exterior face 210 of the electrically insulating sheath 218, e.g., over a portion of the sheath 218. The curved portion 216 is in electrical communication with the distal end 214 of the electrically conductive shaft. The curved portion 216 includes a proximal end 224, a distal end 226, and a central region extending therebetween. Only the proximal end 224 and the distal end 226 of the curved portion 216 are in electrical contact with the distal end 214 of the electrically conductive shaft, the central region being raised above and extending over and across a portion of the insulating sheath 218. In this embodiment, only exposed surfaces of the curved portion 216 of the distal end 214 of the electrically conductive shaft are adapted for applying electrical current to the patient's tissue. The portion beneath the arcuately extending central region of the curved portion 216 may be at least partially covered by the sheath 218.

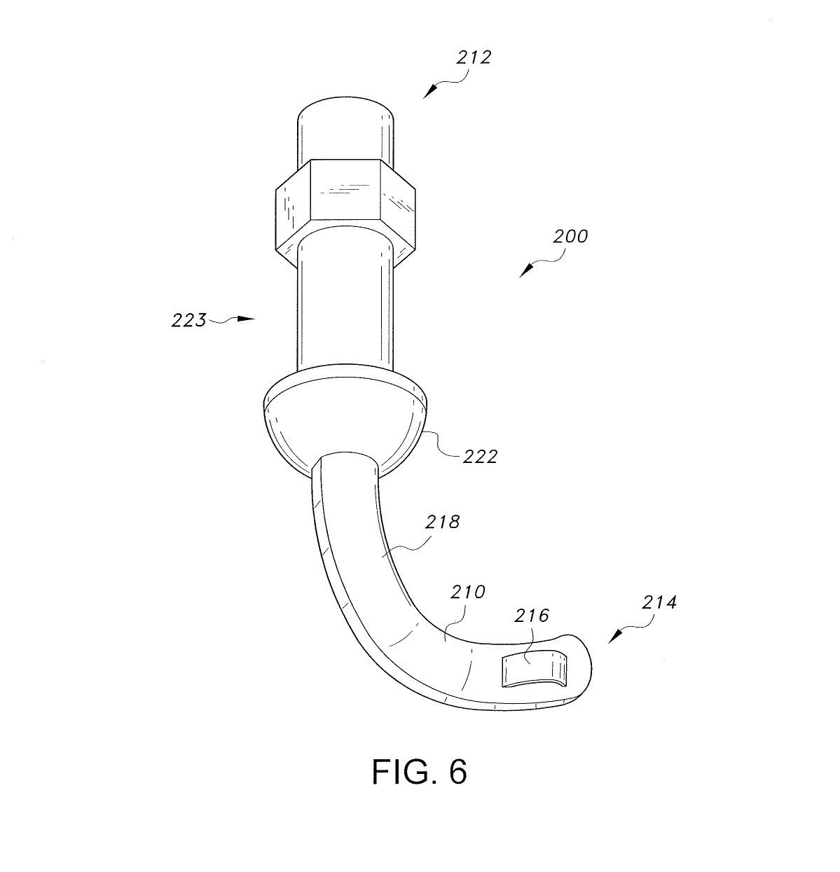

In use, the curved portion 216 is exposed for applying the RF alternating polarity, electrical current to the patient's tissue. Similar to the previous embodiment, although shown as having a pair of flanges 220, 222 mounted on a central portion 223, it should be understood that the overall configuration of active electrode 200 is shown for exemplary purposes only, and that the general shape, contouring and relative dimensions may be varied. For example, some electrosurgical procedures may require the distal end 214 to be curved, as shown in FIG. 6.

It is to be understood that the active electrode assembly for an electrosurgical device is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. An active electrode assembly for an electrosurgical device, comprising:
an electrically conductive shaft having opposed proximal and distal ends, the proximal end being adapted for connection to an electrosurgical device; and
an electrically insulating sheath partially covering the distal end of the electrically conductive shaft, the electrically insulating sheath having a recess formed therein, a portion of the distal end of the electrically conductive shaft being exposed through the recess to define an application surface for applying electrical current to a patient's tissue, wherein the application surface consists of an upper and forward surface of the shaft, further wherein the upper surface of the application surface has first and second laterally opposed sides, each of the laterally opposed sides being totally covered by the electrically insulating sheath thereby leaving only the upper and forward surfaces free and exposed.

2-4. (canceled)

5. An active electrode assembly for an electrosurgical device, comprising:
an electrically conductive shaft having opposed proximal and distal ends, the proximal end being adapted for connection to an electrosurgical device;
a curved application member secured to, and in electrical communication with, the distal end of the electrically conductive shaft; and
an electrically insulating sheath at least partially covering the distal end of the electrically conductive shaft, the curved application member having a curved portion raised above and extending over a portion of the electrically insulating sheath, the curved portion being adapted for applying electrical current to a patient's tissue.

6. The active electrode assembly as recited in claim 5, wherein the curved application member has opposed proximal and distal ends and a central region extending therebetween defining the curved portion.

7. The active electrode assembly as recited in claim 6, wherein the proximal and distal ends of the curved application member contact, and are in electrical communication with, the electrically conductive shaft.

8. The active electrode assembly as recited in claim 7, wherein a region of the electrically conductive shaft beneath the central region of the curved application member is at least partially covered by the electrically insulating sheath.
